(19) United States
(12) Patent Application Publication
(10) Pub. No.: US 2016/0281090 A1
(43) Pub. Date: Sep. 29, 2016

(54) MODIFIED DNA QUADRUPLEX-FORMING OLIGONUCLEOTIDES AND METHODS OF USE

(71) Applicant: ADVANCED CANCER THERAPEUTICS, LLC, Louisville, KY (US)

(72) Inventor: Gilles H. Tapolsky, Louisville, KY (US)

(73) Assignee: Advanced Cancer Therapeutics, LLC, Louisville, KY (US)

(21) Appl. No.: 15/034,034

(22) PCT Filed: Nov. 6, 2014
(86) PCT No.: PCT/US14/64361

§ 371 Date: May 3, 2016

Related U.S. Application Data
(60) Provisional application No. 61/900,658, filed on Nov. 6, 2013.

(51) Int. Cl. C12N 15/113

(52) U.S. Cl. CPC C12N 15/1135; C12N 15/1136; C12N 2310/351; C12N 2310/151

(57) ABSTRACT

Oligonucleotides according to the following Formula I are provided herein: 5′-Xₐ-Q-Y_{b}-3′ wherein X and Y are each independently selected from the group consisting of a lipid and a polyethylene glycol; Q is a quadruplex-forming guanine-rich promoter gene oligonucleotide (GPGO); a is 0 or 1; and b is 0 or 1, wherein the sum of a+b=1 or 2. Also provided are compositions including Formula I oligonucleotides and methods of their use in inhibiting cell growth, treating cancer, and treating tumors.

### RELATED APPLICATION

This application claims priority under 35 U.S.C. §119(e) to U.S. Provisional Application Ser. No. 61/900,658, filed Nov. 6, 2013, which application is hereby incorporated by reference in its entirety.

### TECHNICAL FIELD

The presently disclosed subject matter relates to 5′ and 3′ modified DNA quadruplex-forming oligonucleotides and their methods of use in inhibiting cellular proliferation and treating cancer.

### BACKGROUND ART

Guanine-rich (G-rich) nucleic acid sequences are capable of forming quadruplex, or four-stranded, conformations. These quadruplex structures are comprised of a series of quartets of hydrogen-bonded guanines, which together create a roughly cubical structure. Many cancer-related genes have quadruplex-forming sequences in their G-rich promoter regions. These genes include, but are not limited to, the c-Myc, c-Myb, VEGF, RET, PDGF-A, Bcl-2, c-Kit, K-ras, Rb and HIF-1α genes. Certain guanine-rich promoter gene oligonucleotides (GPGO) sequences are described in U.S. Pat. No. 8,410,070, issued Apr. 2, 2013 to Miller et al., which is incorporated by reference in its entirety.

Guanine-rich quadruplex-forming genomic sequences provide an important target for methods and compositions that inhibit cell proliferation and induce cell death. The need exists to develop therapeutic oligonucleotides and compositions having improved pharmacokinetic properties for targeting guanine-rich quadruplex-forming promoter sequences.

### SUMMARY OF INVENTION

Modified DNA quadruplex-forming oligonucleotides having improved pharmacokinetic properties are disclosed herein. In one embodiment, an oligonucleotide according to the following Formula I is provided:

5′-Xₐ-Q-Y_{b}-3′ Formula I

wherein:

X and Y are each independently selected from the group consisting of a lipid and a polyethylene glycol;

Q is a quadruplex-forming guanine-rich promoter gene oligonucleotide (GPGO);

a is 0 or 1; and

b is 0 or 1, wherein the sum of a+b=1 or 2.

In another embodiment, a pharmaceutical composition comprising an oligonucleotide according to Formula I and a carrier is provided.

In another embodiment, a method of inhibiting cell growth is provided, the method comprising contacting the cell with an oligonucleotide according to Formula I.

In yet another embodiment, a method of treating cancer is provided, the method comprising administering to a patient in need thereof an oligonucleotide according to Formula I.

In another embodiment, a method of treating a patient having a tumor is provided, the method comprising (a) performing a biopsy on the tumor; (b) determining a gene expression profile of the tumor; (c) identifying one or more oncogenes that are overexpressed in the tumor, based on the gene expression profile of step (b); and (d) administering to the patient an oligonucleotide according to Formula I, wherein Q is a GPGO derived from the one or more overexpressed oncogenes identified in step (c).

These and other objects, features, embodiments, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified.

### BRIEF DESCRIPTION OF DRAWINGS

FIG. 1 shows the circular dichroism (CD) spectra for oligonucleotides 1-4, each of which comprises SEQ ID NO: 1 modified at the 5′ end with a lipid or a polyethylene glycol moiety. Results show the modified oligonucleotides retain quadruplex-forming properties.

### DESCRIPTION OF EMBODIMENTS

The details of one or more embodiments of the presently-disclosed subject matter are set forth in this document. Modifications to embodiments described in this document, and other embodiments, will be evident to those of ordinary skill in the art after a study of the information provided herein.

While the following terms are believed to be well understood by those of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.”

Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The term “patient,” as used herein, refers to any mammalian subject, including humans.

The term “safe and effective amount” refers to an amount of a composition high enough to significantly positively modify the symptoms and/or condition to be treated, such as by inhibiting or reducing the proliferation of, or inducing cell death (for example, by inducing apoptosis) of dysplastic, hyperproliferative, or malignant cells, but low enough to avoid serious side effects (at a reasonable risk/benefit ratio), within the scope of sound medical judgment. The safe and effective amount of oligonucleotides for use in the compositions and methods of the invention herein will vary with the particular condition being treated, the age and physical condition of the patient to be treated, the severity of the condition, the duration of the treatment, the nature of concurrent therapy, the particular oligonucleotide(s) being employed, the particular pharmaceutically-acceptable carriers utilized, and like factors within the knowledge and expertise of the attending physician.

As used herein, the term “oligonucleotide” refers to a molecule comprising two or more deoxyribonucleotides or ribonucleotides. The exact size depends on a number of factors including the specificity and binding affinity to target ligands. In referring to “bases” or “nucleotides,” the terms include both deoxyribonucleic acids and ribonucleic acids.

The terms “guanine-rich promoter gene oligonucleotide,” “G-rich promoter gene oligonucleotide,” or “GPGO,” as used herein, refer to oligonucleotides that include the G-rich promoter sequences of oncogenes, such as c-Myc, VEGF, Bcl-2, K-ras, HIF-1α, c-Myb, RET, PDGF-A, c-Kit, and Rb, which form at least one quadruplex, and any oligonucleotide which includes a sequence having at least 80% nucleic acid sequence identity with the G-rich promoter sequence of oncogenes such as c-Myc, VEGF, Bcl-2, K-ras, HIF-1α, c-Myb, RET, PDGF-A, c-Kit, and Rb, and which forms at least one quadruplex. Quadruplex formation may be determined by circular dichroism (CD) spectroscopy. In one embodiment, a GPGO includes a sequence having at least 85%, 90%, 95%, 96%, 97%, 98%, 99%, or essentially 100% nucleic acid sequence identity with the G-rich promoter sequence of oncogenes such as c-Myc, VEGF, Bcl-2, K-ras, HIF-1α, c-Myb, RET, PDGF-A, c-Kit, and Rb.

The term “quadruplex,” as used herein, refers to nucleic acid sequences capable of forming four-stranded conformations. These quadruplex structures are comprised of a series of quartets of hydrogen-bonded guanines, which together create a roughly cubical structure. Many cancer-related genes have quadruplex forming sequences in their G-rich promoter regions. These genes include, but are not limited to, the c-Myc, c-Myb, VEGF, RET, PDGF-A, Bcl-2, c-Kit, BCL-1, K-ras, Rb and HIF-1α genes.

“Percent (%) nucleic acid sequence identity” with respect to sequences identified herein is defined as the percentage of nucleotides in a candidate sequence that are identical with the nucleotides in the sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software.

The term “carrier,” as used herein, includes pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol, and PLURONICS™.

The terms “treat,” “treatment,” and “treating,” as used herein, refer to a method of alleviating or abrogating a disease, disorder, and/or symptoms thereof.

The terms “inhibiting cell development” or “inhibiting cell growth” refer to inhibiting growth of a cell, especially a cancer cell overexpressing any of the genes identified herein, either in vitro or in vivo. Inhibiting cell growth or cell development includes blocking cell cycle progression (at a place other than S phase), for example, G1-arrest or M-phase arrest.

The term “gene expression profile,” as used herein, refers to the measurement of the activity of multiple genes at once to create a global picture of cellular function.

The term “overexpress” or “overexpressed” as used herein refer to a gene product which is expressed at levels greater than normal endogenous expression for that gene product.

A “chemotherapeutic agent” is a chemical compound useful in the treatment of cancer. Examples of chemotherapeutic agents include, but are not limited to, adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol®, Bristol-Myers Squibb Oncology, Princeton, N.J.), and docetaxel (Taxotere®, Rhone-Poulenc Rorer, Antony, France), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, caminomycin, aminopterin, dactinomycin, mitomycins, esperamicins (see U.S. Pat. No. 4,675,187), melphalan and other related nitrogen mustards. Also included in this definition are hormonal agents that act to regulate or inhibit hormone action on tumors such as tamoxifen and onapristone. Suitable chemotherapeutic agents are listed by the National Cancer Institute at the National Institutes of Health, “A to Z List of Cancer Drugs,” available at http://www.cancer.gov/cancertopics/druginfo/alphalist (last accessed Oct. 28, 2014), incorporated herein by reference in its entirety.

### Oligonucleotides and Compositions

In one embodiment, an oligonucleotide according to the following Formula I is provided:

5′-Xₐ-Q-Y_{b}-3′ Formula I

wherein:

X and Y are each independently selected from the group consisting of a lipid and a polyethylene glycol;

Q is a quadruplex-forming guanine-rich promoter gene oligonucleotide (GPGO);

a is 0 or 1; and

b is 0 or 1,

wherein the sum of a+b=1 or 2.

In a specific embodiment, X and Y are independently selected from the sequence modifiers set forth in Table 1, below.

TABLE 1
Sequence Modifiers

| Short | | |
|---|---|---|
| hand | | |
| notation | Chemical Name | Structure |
| Toco | (2R)-2,5,7,8- tetramethyl- 2-[(4R,8R)- (4,8,12- trimethyltridecyl)]- chroman-6-yl-[(2- cyanoethyl)- (N,N- diisopropyl)]- | 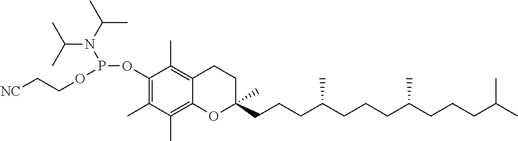 [SMILES: Cc1c(C)c2c(c(C)c1OP(OCCC#N)N(C(C)C)C(C)C)CC[C@@](C)(CCC[C@H](C)CCC[C@H](C)CCCC(C)C)O2] |
| | phosphoramidite | |
| Toco-C8 | 2-Cyanoehtyl (8-(((2R)- 2,5,7,8- tetramethyl-2- [(4R,8R)- (4,8,12- trimethyltridecyl)]- chroman-6- | 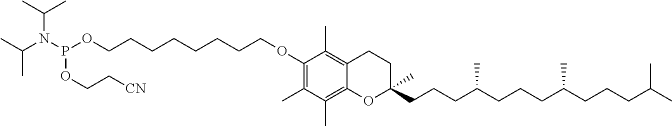 [SMILES: Cc1c(C)c2c(c(C)c1OCCCCCCCCOP(OCCC#N)N(C(C)C)C(C)C)CC[C@@](C)(CCC[C@H](C)CCC[C@H](C)CCCC(C)C)O2] |
| | yl)oxy)octyl) | |
| | diisopropylphos- | |
| | phoramidite | |
| Palmi | 2-cyanoethyl (6- palmitamidohexyl) diisopropylphos- phoramidite | 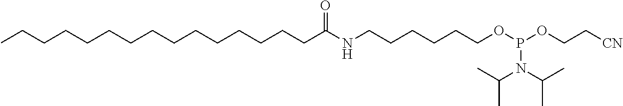 [SMILES: CCCCCCCCCCCCCCCC(=O)NCCCCCCOP(OCCC#N)N(C(C)C)C(C)C] |
| PegS | Polyethyleneglycol phosphoramidite, n = 8 | 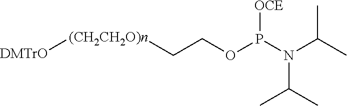 |
| PegL | Polyethyleneglycol phosphoramidite, n = 20 | 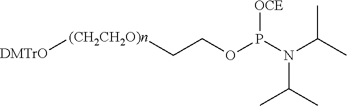 |
| Palmi2 | 5-O-(4,4′- dimethoxytrityl)- 1-O-a-(6- (palmitoylamino)- hexyl)- 2-deoxy-D- ribose-3-O- succinoyl- long-chain alkylamino- CPG | 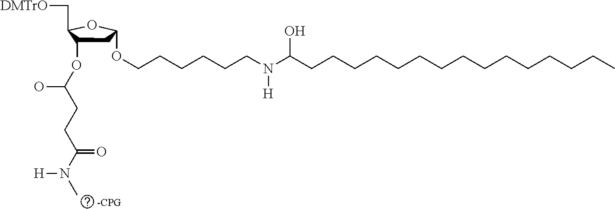 |

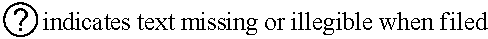

It will be appreciated that, with respect to polyethylene glycol phosphoramidite, having the following structure:

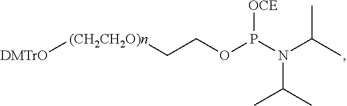

that the molecule can vary in length, such that n is an integer selected from the group consisting of 1, 2, 3, 4, 5, 6, 7, 8, 9, 10, 11, 12, 13, 14, 15, 16, 17, 18, 19, and 20. In a specific embodiment, n is an integer from 1 to 20, or from any subset of integers within that range. In another specific embodiment, n is an integer from 8 to 20.

It will be further appreciated that the oligonucleotides presently disclosed can be modified at the 5′ end, the 3′ end, or both the 5′ and 3′ ends.

In a specific embodiment, X is selected from the sequence modifiers set forth in Table 2, below.

TABLE 2
5′ Sequence Modifiers (X)

| Short | | |
|---|---|---|
| hand | | |
| notation | Chemical Name | Structure |
| Toco | (2R)-2,5,7,8- tetramethyl-2- [(4R,8R)- (4,8,12- trimethyltridecyl)]- chroman- 6-yl-[(2- cyanoethyl)- (N,N- diisopropyl)]- phosphoramidite | 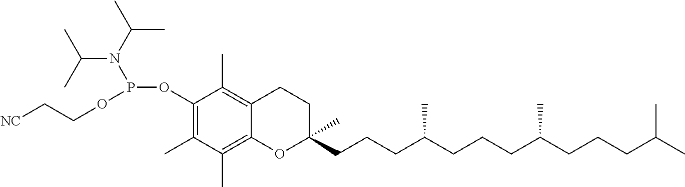 [SMILES: Cc1c(C)c2c(c(C)c1OP(OCCC#N)N(C(C)C)C(C)C)CC[C@@](C)(CCC[C@H](C)CCC[C@H](C)CCCC(C)C)O2] |
| Toco-C8 | 2-Cyanoehtyl (8-(((2R)- 2,5,7,8- tetramethyl-2- [(4R,8R)- (4,8,12- trimethyltridecyl)]- chroman-6- | 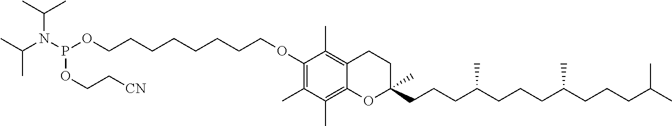 [SMILES: Cc1c(C)c2c(c(C)c1OCCCCCCCCOP(OCCC#N)N(C(C)C)C(C)C)CC[C@@](C)(CCC[C@H](C)CCC[C@H](C)CCCC(C)C)O2] |
| | yl)oxy)octyl) | |
| | diisopropylphos- | |
| | phoramidite | |
| Palmi | 2-cyanoethyl (6- palmitamidohexyl) diisopropylphos- phoramidite | 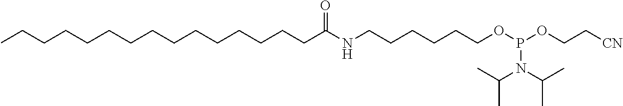 [SMILES: CCCCCCCCCCCCCCCC(=O)NCCCCCCOP(OCCC#N)N(C(C)C)C(C)C] |
| PegS | Polyethyleneglycol phosphoramidite, n = 8 | 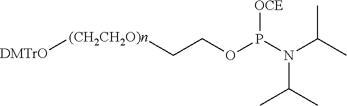 |
| PegL | Polyethyleneglycol phosphoramidite, n = 20 | 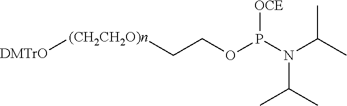 |

In another specific embodiment, Y is selected from the sequence modifier set forth in Table 3, below:

TABLE 3
3′ Sequence Modifier (Y)

| Short hand | | |
|---|---|---|
| notation | Chemical Name | Structure |
| Palmi2 | 5-O-(4,4′- dimethoxytrityl)-1- O-a-(6- (palmitoylamino)- hexyl)-2-deoxy-D- ribose-3-O- succinoyl-long-chain alkylamino-CPG | 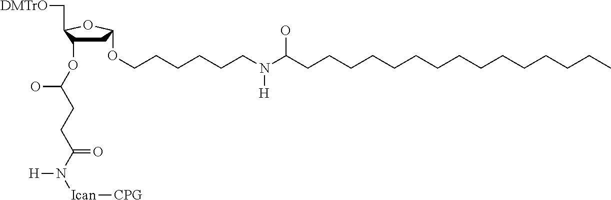 |

In another specific embodiment of Formula I oligonucleotides, X is selected from the group consisting of a lipid and a polyethylene glycol; and Y is a lipid.

In a specific embodiment, Q is a GPGO derived from an oncogene selected from the group consisting of c-Myc, c-Myb, VEGF, Bcl-2, K-ras, HIF-1α, Rb, RET, c-Kit, and PDGF-A. In one embodiment, Q is a DNA sequence selected from the group consisting of SEQ ID NOs: 1-70.

In a specific embodiment, Q is a c-Myc sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 14, or a sequence having at least 85%, 90%, 95%, 96%, 97%, 98%, 99%, or essentially 100% nucleic acid sequence identity with a c-Myc sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 14.

In another embodiment, Q is a c-Myb sequence selected from the group consisting of SEQ ID NO: 15, SEQ ID NO: 16, SEQ ID NO: 17, SEQ ID NO: 18, SEQ ID NO: 19, and SEQ ID NO: 20, or a sequence having at least 85%, 90%, 95%, 96%, 97%, 98%, 99%, or essentially 100% nucleic acid sequence identity with a c-Myb sequence selected from the group consisting of SEQ ID NO: 15, SEQ ID NO: 16, SEQ ID NO: 17, SEQ ID NO: 18, SEQ ID NO: 19, and SEQ ID NO: 20.

In another embodiment, Q is a VEGF sequence selected from the group consisting of SEQ ID NO: 21, SEQ ID NO: 22, SEQ ID NO: 23, SEQ ID NO: 24, SEQ ID NO: 25, and SEQ ID NO: 26, or a sequence having at least 85%, 90%, 95%, 96%, 97%, 98%, 99%, or essentially 100% nucleic acid sequence identity with a VEGF sequence selected from the group consisting of SEQ ID NO: 21, SEQ ID NO: 22, SEQ ID NO: 23, SEQ ID NO: 24, SEQ ID NO: 25, and SEQ ID NO: 26.

In another embodiment, Q is a K-ras sequence selected from the group consisting of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35, or a sequence having at least 85%, 90%, 95%, 96%, 97%, 98%, 99%, or essentially 100% nucleic acid sequence identity with a K-ras sequence selected from the group consisting of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35.

In another embodiment, Q is a RET sequence selected from the group consisting of SEQ ID NO: 36, SEQ ID NO: 37, SEQ ID NO: 38, SEQ ID NO: 39, and SEQ ID NO: 40, or a sequence having at least 85%, 90%, 95%, 96%, 97%, 98%, 99%, or essentially 100% nucleic acid sequence identity with a RET sequence selected from the group consisting of SEQ ID NO: 36, SEQ ID NO: 37, SEQ ID NO: 38, SEQ ID NO: 39, and SEQ ID NO: 40.

In another embodiment, Q is a HIF-1α sequence selected from the group consisting of SEQ ID NO: 41, SEQ ID NO: 42, SEQ ID NO: 43, SEQ ID NO: 44, SEQ ID NO: 45, SEQ ID NO: 46, and SEQ ID NO: 47, or a sequence having at least 85%, 90%, 95%, 96%, 97%, 98%, 99%, or essentially 100% nucleic acid sequence identity with a HIF-1α sequence selected from the group consisting of SEQ ID NO: 41, SEQ ID NO: 42, SEQ ID NO: 43, SEQ ID NO: 44, SEQ ID NO: 45, SEQ ID NO: 46, and SEQ ID NO: 47.

In another embodiment, Q is a PDGF-A sequence selected from the group consisting of SEQ ID NO: 48, SEQ ID NO: 49, SEQ ID NO: 50, SEQ ID NO: 51, SEQ ID NO: 52, SEQ ID NO: 53, SEQ ID NO: 54, and SEQ ID NO: 55, or a sequence having at least 85%, 90%, 95%, 96%, 97%, 98%, 99%, or essentially 100% nucleic acid sequence identity with a PDGF-A sequence selected from the group consisting of SEQ ID NO: 48, SEQ ID NO: 49, SEQ ID NO: 50, SEQ ID NO: 51, SEQ ID NO: 52, SEQ ID NO: 53, SEQ ID NO: 54, and SEQ ID NO: 55.

In another embodiment, Q is a Bcl-2 sequence selected from the group consisting of SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, and SEQ ID NO: 65, or a sequence having at least 85%, 90%, 95%, 96%, 97%, 98%, 99%, or essentially 100% nucleic acid sequence identity with a Bcl-2 sequence selected from the group consisting of SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, and SEQ ID NO: 65.

In another embodiment, Q is a c-Kit sequence selected from the group consisting of SEQ ID NO: 66, SEQ ID NO: 67, SEQ ID NO: 68, SEQ ID NO: 69, and SEQ ID NO: 70, or a sequence having at least 85%, 90%, 95%, 96%, 97%, 98%, 99%, or essentially 100% nucleic acid sequence identity with a c-Kit sequence selected from the group consisting of SEQ ID NO: 66, SEQ ID NO: 67, SEQ ID NO: 68, SEQ ID NO: 69, and SEQ ID NO: 70.

### Dosage Forms and Administration

Provided herein are compositions comprising a safe and effective amount of an oligonucleotide according to Formula I and a carrier, wherein the oligonucleotide forms at least one quadruplex.

It will be appreciated that the Formula I oligonucleotides and compositions disclosed herein can be administered to a patient or subject either alone or as part of a pharmaceutical composition. The Formula I oligonucleotides can be administered to patients either orally, rectally, parenterally (intravenously, intramuscularly, or subcutaneously), intracistemally, intravaginally, intraperitonally, intravesically, locally (powders, ointments, or drops), or as a buccal or nasal spray.

Compositions comprising the Formula I oligonucleotides of the present invention suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active ingredient is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethylene glycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

Besides such inert diluents, the compositions can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal or vaginal administrations are preferably suppositories which can be prepared by mixing the compounds of the present invention with suitable non-irritating excipients or carriers such as cocoa butter, polyethylene glycol or a suppository wax, which are solid at ordinary temperatures but liquid at body temperature and therefore, melt in the rectum or vaginal cavity and release the active component.

Dosage forms for topical administration of a Formula I oligonucleotide of the present invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

In addition, the oligonucleotides of the present invention can exist in unsolvated as well as solvated forms with pharmaceutically acceptable solvents such as water, ethanol, and the like. In general, the solvated forms are considered equivalent to the unsolvated forms for the purposes of the present invention.

The Formula I oligonucleotides of the present invention can be administered to a patient at dosage levels in the range of about 1.5 mg to about 150 mg per day; it is also possible to administer larger amounts, such as from about 150 mg to 1 g per day. A unit dosage form of Formula I oligonucleotides is an amount which would be administered as a single dose. For a normal human adult having a body weight of about 70 kilograms, a dosage in the range of about 0.2 mg to about 2.0 mg per kilogram of body weight per day is suitable. The specific dosage used, however, can vary. For example, the dosage can depend on a number of factors including the requirements of the patient, the severity of the condition being treated, and the pharmacological activity of the compound being used. The determination of optimum dosages for a particular patient is well known to those skilled in the art. The Formula I oligonucleotides of the present invention can be given in single and/or multiple dosages.

The Formula I oligonucleotides disclosed herein are synthesized using standard organic synthetic techniques known to the ordinary skilled artisan.

The Formula I oligonucleotides of the present invention may also be used in combination with other chemotherapeutic agents to provide a synergistic or enhanced efficacy or inhibition of neoplastic cell growth. For example, the Formula I oligonucleotides of the present invention can be administered in combination with chemotherapeutic agents including, for example, cis-platin, mitoxantrone, etoposide, camptothecin, 5-fluorouracil, vinblastine, paclitaxel, docetaxel, mithramycin A, dexamethasone, caffeine, and other chemotherapeutic agents and/or growth inhibitory agents well known to those skilled in the art. In certain embodiments, a Formula I oligonucleotide is administered together with a chemotherapeutic agent in the same unit dosage. In other embodiments, a Formula I oligonucleotide and a chemotherapeutic agent are administered in separate dosage forms, concurrently or consecutively.

### Methods of Use

In one embodiment, a method of inhibiting cell growth is provided, the method comprising contacting a cell with a Formula I oligonucleotide or a composition comprising a Formula I oligonucleotide.

In another embodiment, a method of treating cancer is provided, the method comprising administering to a patient in need thereof a Formula I oligonucleotide or a composition comprising a Formula I oligonucleotide.

In certain embodiments, the cancer to be treated is a cancer that expresses an oncogene selected from the group consisting of c-Myc, c-Myb, VEGF, Bcl-2, K-ras, HIF-1α, Rb, RET, c-Kit, and PDGF-A. In a specific embodiment, the cancer to be treated is a cancer selected from the group consisting of leukemia, lymphoma, brain cancer, breast cancer, lung cancer, pancreatic cancer, colon cancer, ovarian cancer, liver cancer, prostate cancer, bone cancer, melanoma, and the like.

In another embodiment, a method of treating a patient having a tumor is provided, the method comprising: (a) performing a biopsy on the tumor; (b) determining a gene expression profile of the tumor; (c) identifying one or more oncogenes that are overexpressed in the tumor, based on the gene expression profile of step (b); (d) administering to the patient a composition according to Formula I, wherein Q is a GPGO derived from the one or more overexpressed oncogenes identified in step (c). In this way, the tumor therapy is specifically selected for the gene expression profile of the particular tumor to be treated. The skilled artisan will appreciate that a gene expression profile can be determined using a variety of techniques known in the art, including but not limited to, DNA and RNA microarray technologies, SAGE (serial analysis of gene expression), Western and Northern blots, and reverse transcription polymerase chain reaction (RT-PCR).

### EXAMPLES

The following examples are given by way of illustration and are in no way intended to limit the scope of the present invention.

### Example 1

Representative oligonucleotides of Formula I are set forth in the following Tables 4-13. It is noted that the 5′ X and 3′ Y sequence modifiers are given by way of illustration and are not intended to limit the scope of the presently disclosed oligonucleotides.

TABLE 4
Modified Oligonucleotide Sequences Targeting c-Myc

| | Q = SEQ | | |
|---|---|---|---|
| Oligo Identifier | ID NO: | X | Y |
| 1 | 1 | Toco | — |
| 2 | 1 | Toco-C8 | — |
| 3 | 1 | Palmi | — |
| 4 | 1 | PegS | — |
| 5 | 1 | PegL | — |
| 6 | 2 | Palmi | — |
| 7 | 3 | Palmi | — |
| 8 | 4 | Palmi | — |
| 9 | 5 | Palmi | — |
| 10 | 6 | Palmi | — |
| 11 | 7 | Palmi | — |
| 12 | 8 | Palmi | — |
| 13 | 9 | Palmi | — |
| 14 | 10 | Palmi | — |
| 15 | 11 | Palmi | — |
| 16 | 3 | Palmi | — |
| 17 | 12 | Palmi | — |
| 18 | 13 | Palmi | — |
| 19 | 14 | Palmi | — |
| 20 | 1 | — | Palmi2 |

TABLE 5
Modified Oligonucleotide Sequences Targeting c-Myb

| | Q = SEQ | | |
|---|---|---|---|
| Oligo Identifier | ID NO: | X | Y |
| 21 | 15 | Toco | — |
| 22 | 15 | Toco-C8 | — |
| 23 | 15 | Palmi | — |
| 24 | 15 | PegS | — |
| 25 | 15 | PegL | — |
| 26 | 16 | Palmi | — |
| 27 | 17 | Palmi | — |
| 28 | 18 | Palmi | — |
| 29 | 19 | Palmi | — |
| 30 | 20 | Palmi | — |
| 31 | 15 | — | Palmi2 |

TABLE 6
Modified Oligonucleotide Sequences Targeting VEGF

| | Q = SEQ | | |
|---|---|---|---|
| Oligo Identifier | ID NO: | X | Y |
| 32 | 21 | Toco | — |
| 33 | 21 | Toco | — |
| 34 | 22 | Toco | — |
| 35 | 23 | Toco | — |
| 36 | 24 | Toco | — |
| 37 | 25 | Toco | — |
| 38 | 26 | Toco | — |
| 39 | 21 | — | Palmi2 |

TABLE 7
Modified Oligonucleotide Sequences Targeting K-ras

| | Q = SEQ | | |
|---|---|---|---|
| Oligo Identifier | ID NO: | X | Y |
| 40 | 27 | Toco | — |
| 41 | 27 | Palmi | — |
| 42 | 28 | Toco | — |
| 43 | 29 | Toco | — |
| 44 | 30 | Toco | — |
| 45 | 31 | Toco | — |
| 46 | 32 | Toco | — |
| 47 | 33 | Toco | — |
| 48 | 34 | Toco | — |
| 49 | 35 | Toco | — |
| 50 | 27 | — | Palmi2 |

TABLE 8
Modified Oligonucleotide Sequences Targeting RET

| | Q = SEQ | | |
|---|---|---|---|
| Oligo Identifier | ID NO: | X | Y |
| 51 | 36 | Toco | — |
| 52 | 37 | Toco | — |
| 53 | 38 | Palmi | — |
| 54 | 39 | PegS | — |
| 55 | 40 | PegL | — |
| 56 | 36 | — | Palmi2 |

TABLE 9
Modified Oligonucleotide Sequences Targeting HIF-1α

| | Q = SEQ | | |
|---|---|---|---|
| Oligo Identifier | ID NO: | X | Y |
| 57 | 41 | Palmi | — |
| 58 | 42 | Palmi | — |
| 59 | 43 | Palmi | — |
| 60 | 44 | Palmi | — |
| 61 | 45 | Palmi | — |
| 62 | 46 | Palmi | — |
| 63 | 47 | Palmi | — |
| 64 | 41 | — | Palmi2 |

TABLE 10
Modified Oligonucleotide Sequences Targeting PDGF-A

| | Q = SEQ | | |
|---|---|---|---|
| Oligo Identifier | ID NO: | X | Y |
| 65 | 48 | Toco | — |
| 66 | 49 | Toco | — |
| 67 | 50 | Toco | — |
| 68 | 51 | Toco | — |
| 69 | 52 | Toco | — |
| 70 | 53 | Toco | — |
| 71 | 54 | Toco | — |
| 72 | 55 | Toco | — |
| 73 | 48 | — | Palmi2 |

TABLE 11
Modified Oligonucleotide Sequences Targeting Bcl-2

| | Q = SEQ | | |
|---|---|---|---|
| Oligo Identifier | ID NO: | X | Y |
| 74 | 56 | Palmi | — |
| 75 | 57 | Palmi | — |
| 76 | 58 | Palmi | — |
| 77 | 59 | Palmi | — |
| 78 | 60 | Toco | — |
| 79 | 61 | Toco | — |
| 80 | 62 | Toco | — |
| 81 | 63 | Toco | — |
| 82 | 64 | Toco | — |
| 83 | 65 | Toco | — |
| 84 | 58 | — | Palmi2 |

TABLE 12
Modified Oligonucleotide Sequences Targeting c-Kit

| | Q = SEQ | | |
|---|---|---|---|
| Oligo Identifier | ID NO: | X | Y |
| 85 | 66 | Toco | — |
| 86 | 67 | Toco | — |
| 87 | 68 | Toco | — |
| 88 | 69 | Toco | — |
| 89 | 70 | Toco | — |
| 90 | 66 | — | Palmi2 |

### Example 2

The ability of the Formula I oligonucleotides to kill or inhibit the proliferation of cancer cells was measured using either the MTT assay, the Alamar Blue® assay (Invitrogen, Grand Island, N.Y.), or the CellTiter Glo™ assay (Promega, Madison, Wis.) using 72 or 144 hours exposure. MTT assay results for different cancer cell lines are shown in Tables 13-16 below and demonstrate that these oligonucleotides inhibit cancer cell proliferation in many types of cancer cell lines.

A typical experiment is as follows. Cells of the desired tumor cell line are plated at 2×10⁵ cells/ml in 96 well plates. Twice the indicated concentrations of the oligonucleotides are added to cells the following day in an equal volume of media. 72 or 144 hours later, cells are lysed and subjected to ATP determination using the CellTiter-Glo™ Luminescent Cell Viability Assay kit. Experiments are performed in triplicate. When using the MTT assay or the AlamarBlue® assay, the experimental conditions are essentially similar; at the end of the incubation period, 20 microliters of the MTT solution is added per well and the samples are incubated for an additional 4 hours, rinsed, and absorbance at 570 nm is measured. Results for the inhibition of cell proliferation are reported as the IC₅₀ (the concentration resulting in 50% inhibition of proliferation of the cell population) and are listed in Tables 13-16 below. Cell lines include lung, colon, prostate, breast, ovarian, and pancreatic cancer lines.

TABLE 13
IC₅₀ values (microM) for c-Myc related sequences

| Oligo | Cell | | | | MV4- |
|---|---|---|---|---|---|
| Identifier | line | Raji | A549 | HL-60 | 11 |
| 1 | | 0.75 ± 0.1 | 5.6 ± 0.2 | 1.7 ± 0.2 | 2.3 ± 0.1 |
| 2 | | 0.55 ± 0.1 | 6.1 ± 0.2 | 1.5 ± 0.1 | 2.0 ± 0.1 |
| 3 | | 0.65 ± 0.1 | 5.3 ± 0.2 | 1.2 ± 0.2 | 1.6 ± 0.1 |
| 4 | | 0.6 ± 0.1 | 2.3 ± 0.2 | 1.2 ± 0.2 | 0.7 ± 0.1 |
| 5 | | 0.6 ± 0.1 | 2.3 ± 0.2 | 1.2 ± 0.2 | 0.7 ± 0.1 |
| 6 | | 0.58 ± 0.1 | | | |
| 7 | | 0.60 ± 0.2 | | | |
| 8 | | 0.62 ± 0.1 | | | |
| 9 | | 0.58 ± 0.1 | | | |
| 10 | | 0.63 ± 0.1 | | | |
| 11 | | 0.76 ± 0.2 | | | |
| 12 | | 0.58 ± 0.2 | | | |
| 13 | | 0.80 ± 0.1 | | | |
| 14 | | 1.1 ± 0.2 | | | |
| 15 | | 0.75 ± 0.1 | | | |
| 16 | | 0.59 ± 0.2 | | | |
| 17 | | 0.58 ± 0.3 | | | |
| 18 | | 0.61 ± 0.1 | | | |
| 19 | | 0.89 ± 0.2 | | | |
| 20 | | 0.78 ± 0.1 | | | |

TABLE 14
IC₅₀ values (microM) for VEGF related sequences.

| Oligo | Cell Line |
|---|---|
| Identifier | A549 |
| 32 | 2.55 ± 0.3 |
| 33 | 1.33 ± 0.2 |
| 36 | 1.6 ± 0.1 |
| 38 | 1.0 ± 0.2 |

TABLE 15
IC₅₀ values (microM) for K-ras related sequences.

| Oligo | Cell Line |
|---|---|
| Identifier | Capan-1 |
| 40 | 1.1 ± 0.3 |
| 42 | 1..8 ± 0.2 |
| 47 | 0.9 ± 0.1 |
| 49 | 1.0 ± 0.2 |

TABLE 16
IC₅₀ values (microM) for Bcl-2 related sequences.

| Oligo | Cell | | | |
|---|---|---|---|---|
| Identifier | line | Raji | A549 | HL-60 |
| 74 | | 1.75 ± 0.2 | 5.6 ± 0.3 | 1.7 ± 0.1 |
| 75 | | 1.55 ± 0.3 | 6.1 ± 0.2 | 1.5 ± 0.1 |
| 76 | | 0.95 ± 0.2 | 5.3 ± 0.3 | 1.2 ± 0.1 |
| 81 | | 1.6 ± 0.2 | 4.9 ± 0.2 | 1.2 ± 0.1 |
| 83 | | 1.5 ± 0.2 | 4.1 ± 0.2 | 1.2 ± 0.1 |

### Example 3

The ability to inhibit cancer cell proliferation is linked to the ability of the presently disclosed sequences to form three dimensional quadruplex DNA structures. In the 220 to 320 nm range of the UV spectrum, circular dichroism (CD) spectra reveal whether modified oligonucleotides adopt a quadruplex structure, as well as the nature of the structure (e.g., parallel, anti-parallel, basket, boat, and the like). See, for example, Karsisiotis, et al., Topological characterization of nucleic acid G-quadruplexes by UV absorption and circular dichroism, Angewandte Chemie 50:10645-48 (2011).

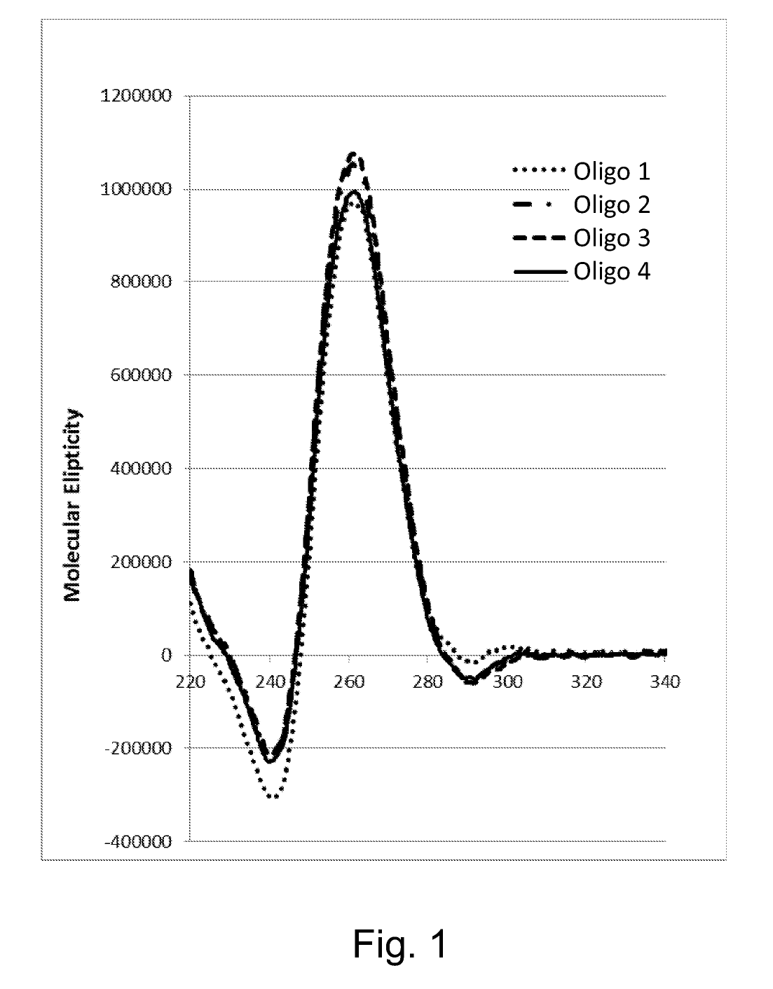

Surprisingly, the inclusion of 3′ and/or 5′ lipid or hydrophilic (polyethylene glycol) modifiers does not adversely impact the quadruplex forming properties of the present Formula I oligonucleotides. As shown in FIG. 1, the oligonucleotide having SEQ ID NO: 1 was modified with various 5′ lipid or polyethylene glycol moieties (oligonucleotides 1-4), yet still retained quadruplex forming properties, as evidenced by the CD spectra of oligonucleotides 1-4.

### Example 4

Pharmacokinetic (PK) parameters were determined in mice following IV administration of modified and unmodified oligonucleotides. Typical study design includes 3 to 6 male or female mice per time point. Mice are 7-8 weeks old NOD, SCID, or similar animal model. Mice are dosed at 100 mg/kg or 50 mg/kg of oligonucleotide in nuclease-free buffer (such as 10 mM potassium buffer). Modified and unmodified oligonucleotides are tested, in order to compare half life in vivo. Blood samples are collected at intervals. Plasma samples are extracted and analyzed using an LC-MS/MS method. Results are shown in Table 17.

Surprisingly, the modified oligonucleotides disclosed herein not only maintain the ability to form quadruplex DNA structures, but also demonstrate improved PK properties. Specifically, half life of terminally modified oligonucleotides is considerably increased (20 to 100 fold longer, depending on the modifier selected and the molecular weight of the oligonucleotide).

TABLE 17
PK parameters (IV dosing; 100 mg/kg; * 50 mg/kg).

| | T_{1/2} | Cₘₐₓ | AUC_{0-inf} | Cl_obs | Vss_obs |
|---|---|---|---|---|---|
| Oligo | (h) | (ng/mL) | (ng · h/ml) | (mL/min/kg) | (L/kg) |
| SEQ ID NO: 1 | 0.11 | 148336 | 34894 | 46.8 | 0.65 |
| SEQ ID NO: 1 | 4.8 | 181436 | 35677 | 46.7 | 7.40 |
| modified at 5′ | | | | | |
| end with PegS | | | | | |
| SEQ ID NO: 1 | 5.1 | 188625 | 36523 | 41.2 | 8.10 |
| modified at 5′ | | | | | |
| end with Palmi | | | | | |
| SEQ ID NO: 1 | 5.6 | 179453 | 34589 | 43.2 | 8.40 |
| modified at 5′ | | | | | |
| end with Toco | | | | | |
| SEQ ID NO: 3 * | 0.12 | 71234 | 19234 | 41.8 | 0.71 |
| SEQ ID NO: 3 | 6.1 | 179189 | 36255 | 43.2 | 7.90 |
| modified at 5′ | | | | | |
| end with PegS | | | | | |
| SEQ ID NO: 6 * | 0.09 | 69345 | 18254 | 43.2 | 0.85 |
| SEQ ID NO: 6 | 5.2 | 181436 | 35677 | 41.6 | 7.86 |
| modified at 5′ | | | | | |
| end with PegS | | | | | |

All documents cited are incorporated herein by reference; the citation of any document is not to be construed as an admission that it is prior art with respect to the present invention.

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

1. An oligonucleotide according to the following Formula I:
5′-Xₐ-Q-Y_{b}-3′ Formula I
wherein:
X and Y are each independently selected from the group consisting of a lipid and a polyethylene glycol;
Q is a quadruplex-forming guanine-rich promoter gene oligonucleotide (GPGO);
a is 0 or 1; and
b is 0 or 1,
wherein the sum of a+b=1 or 2.

2. The oligonucleotide of claim 1, wherein the lipid is selected from the group consisting of

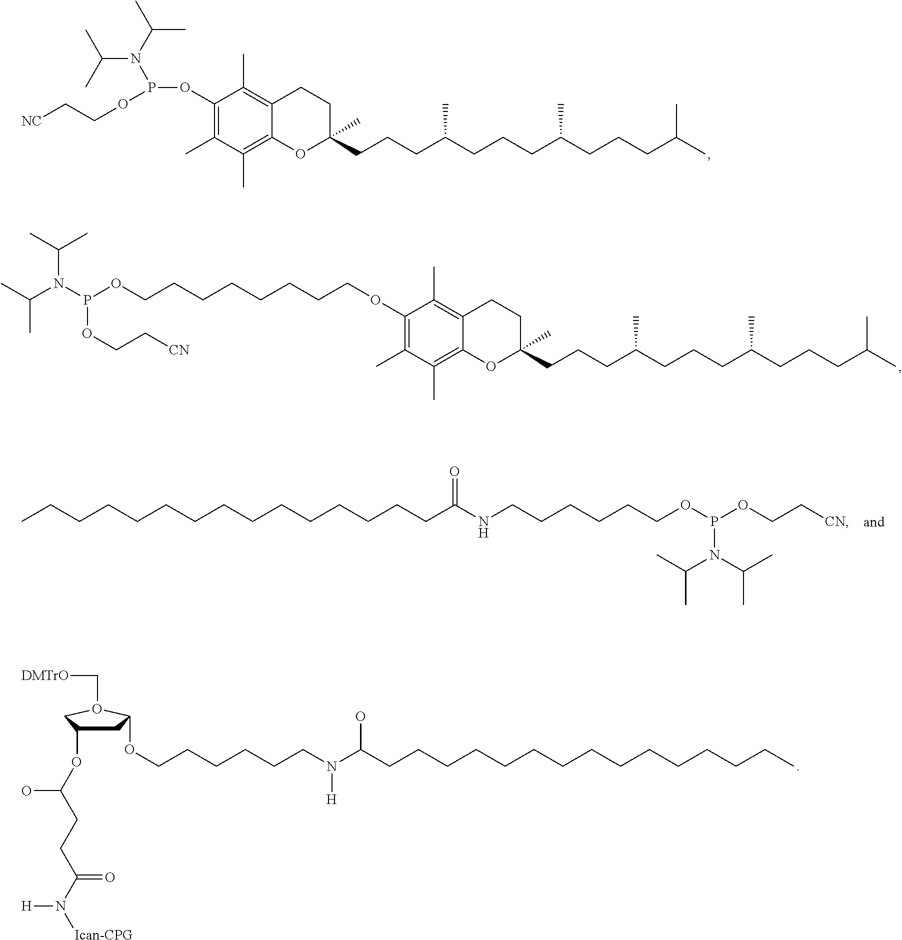

3. The oligonucleotide of claim 1, wherein the polyethylene glycol has the following structure: 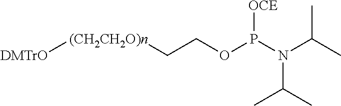
wherein n is 8 or 20.

4. The oligonucleotide of claim 1, wherein X is selected from the group consisting of 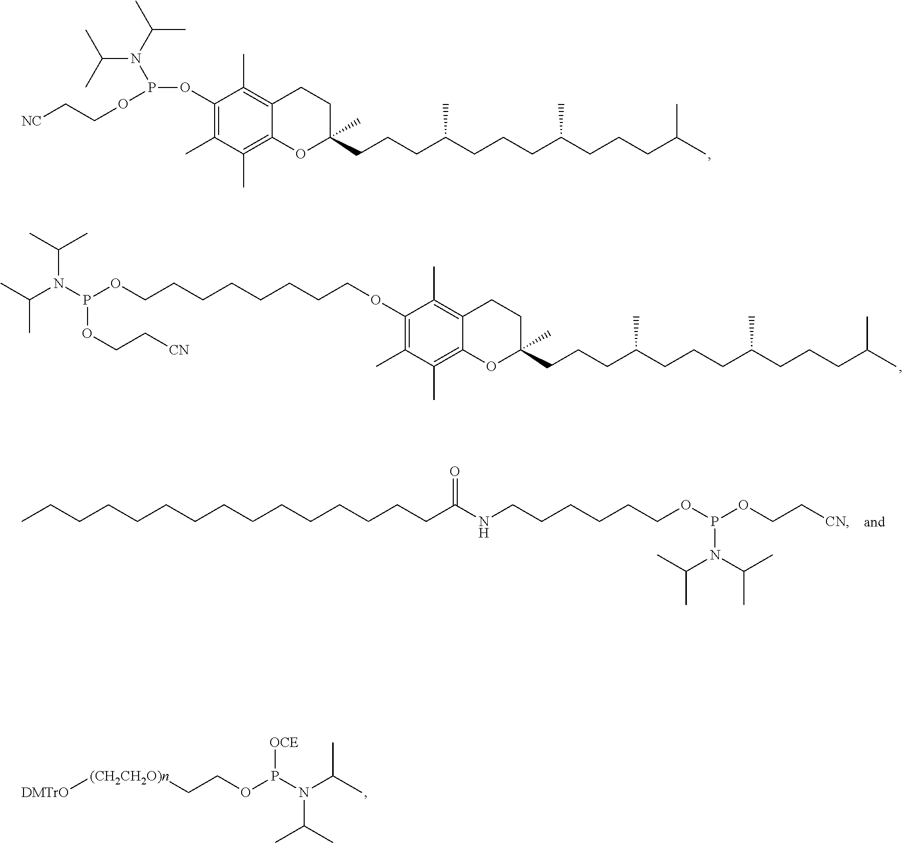
wherein n is 8 or 20; and
wherein Y is

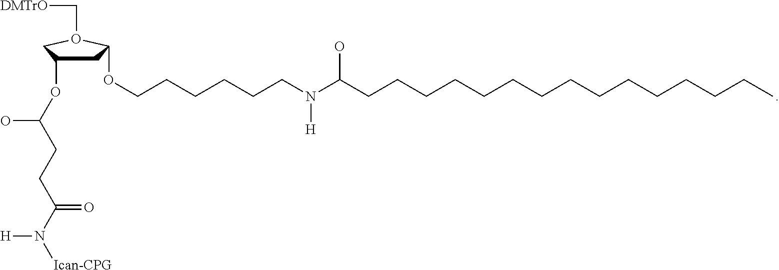

5. The oligonucleotide of claim 4, wherein Q is a GPGO derived from an oncogene selected from the group consisting of c-Myc, c-Myb, VEGF, Bcl-2, K-ras, HIF-1α, Rb, RET, c-Kit, and PDGF-A.

6. The oligonucleotide of claim 5, wherein Q is a c-Myc sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 14; a is 1; and b is 0.

7. The oligonucleotide of claim 5, wherein Q is a c-Myb sequence selected from the group consisting of SEQ ID NO: 15, SEQ ID NO: 16, SEQ ID NO: 17, SEQ ID NO: 18, SEQ ID NO: 19, and SEQ ID NO: 20; a is 1; and b is 0.

8. The oligonucleotide of claim 5, wherein Q is a VEGF sequence selected from the group consisting of SEQ ID NO: 21, SEQ ID NO: 22, SEQ ID NO: 23, SEQ ID NO: 24, SEQ ID NO: 25, and SEQ ID NO: 26;
X is 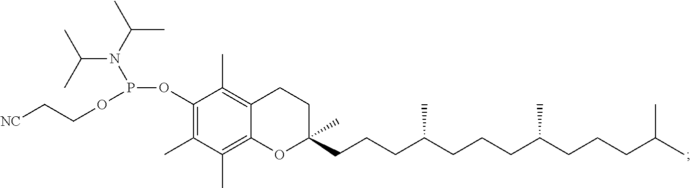 [SMILES: Cc1c(C)c2c(c(C)c1OP(OCCC#N)N(C(C)C)C(C)C)CC[C@@](C)(CCC[C@H](C)CCC[C@H](C)CCCC(C)C)O2]
a is 1; and b is 0.

9. The oligonucleotide of claim 5, wherein Q is a K-ras sequence selected from the group consisting of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35;
X is selected from the group consisting of 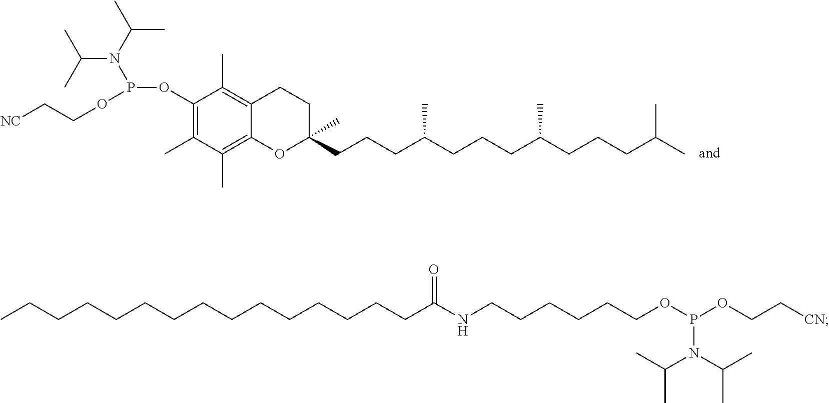
a is 1; and b is 0.

10. The oligonucleotide of claim 5, wherein Q is a RET sequence selected from the group consisting of SEQ ID NO: 36, SEQ ID NO: 37, SEQ ID NO: 38, SEQ ID NO: 39, and SEQ ID NO: 40;
X is selected from the group consisting of 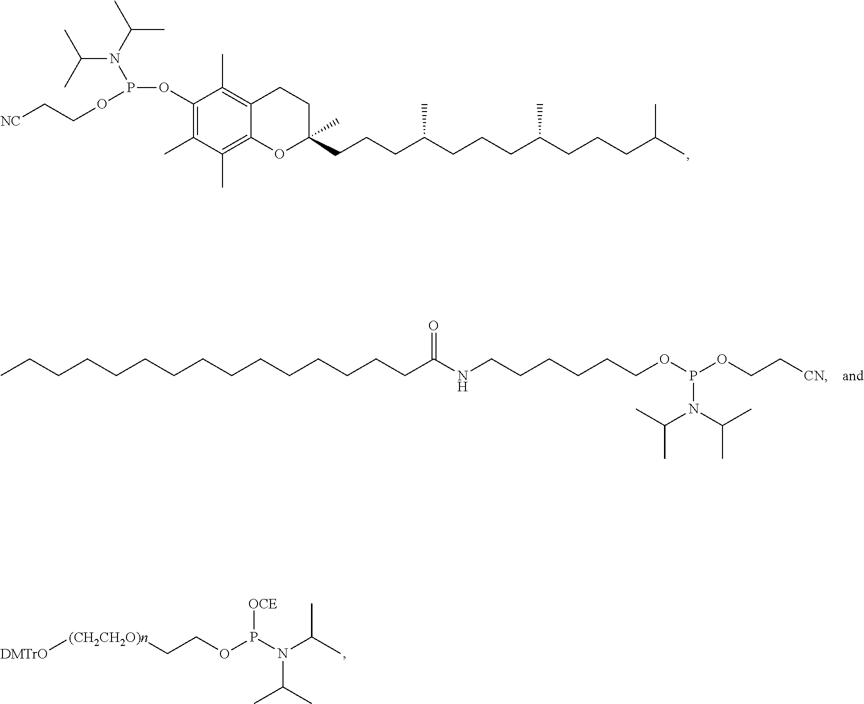
wherein n is 8 or 20; a is 1; and b is 0.

11. The oligonucleotide of claim 5, wherein Q is a HIF-1α sequence selected from the group consisting of SEQ ID NO: 41, SEQ ID NO: 42, SEQ ID NO: 43, SEQ ID NO: 44, SEQ ID NO: 45, SEQ ID NO: 46, and SEQ ID NO: 47;
X is 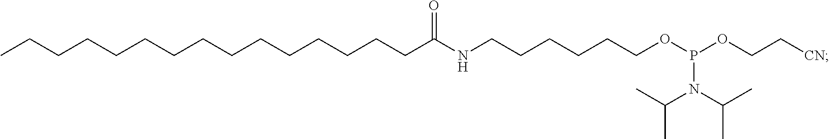 [SMILES: CCCCCCCCCCCCCCCC(=O)NCCCCCCOP(OCCC#N)N(C(C)C)C(C)C]
a is 1; and b is 0.

12. The oligonucleotide of claim 5, wherein Q is a PDGF-A sequence selected from the group consisting of SEQ ID NO: 48, SEQ ID NO: 49, SEQ ID NO: 50, SEQ ID NO: 51, SEQ ID NO: 52, SEQ ID NO: 53, SEQ ID NO: 54, and SEQ ID NO: 55;
X is 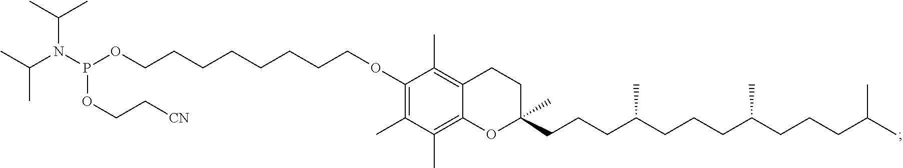 [SMILES: Cc1c(C)c2c(c(C)c1OCCCCCCCCOP(OCCC#N)N(C(C)C)C(C)C)CC[C@@](C)(CCC[C@H](C)CCC[C@H](C)CCCC(C)C)O2]
a is 1; and b is 0.

13. The oligonucleotide of claim 5, wherein Q is a Bcl-2 sequence selected from the group consisting of SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, and SEQ ID NO: 65;
X is selected from the group consisting of 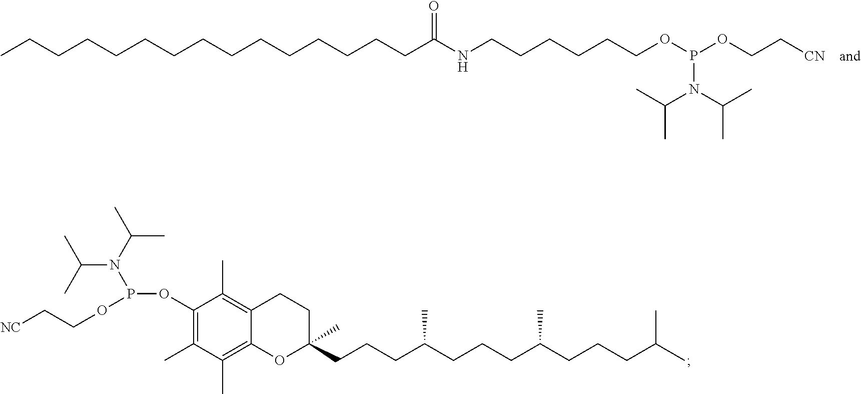 [SMILES: CCCCCCCCCCCCCCCC(=O)NCCCCCCOP(OCCC#N)N(C(C)C)C(C)C ; Cc1c(C)c2c(c(C)c1OP(OCCC#N)N(C(C)C)C(C)C)CC[C@@](C)(CCC[C@H](C)CCC[C@H](C)CCCC(C)C)O2]
a is 1; and b is 0.

14. The oligonucleotide of claim 5, wherein Q is a c-Kit sequence selected from the group consisting of SEQ ID NOs: 66, SEQ ID NO: 67, SEQ ID NO: 68, SEQ ID NO: 69, and SEQ ID NO: 70;
X is 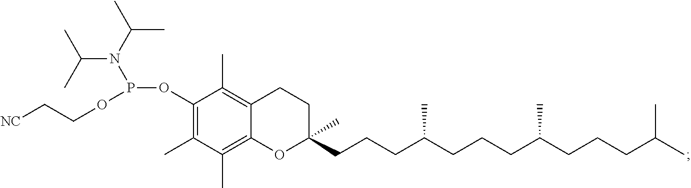 [SMILES: Cc1c(C)c2c(c(C)c1OP(OCCC#N)N(C(C)C)C(C)C)CC[C@@](C)(CCC[C@H](C)CCC[C@H](C)CCCC(C)C)O2]
a is 1; and b is 0.

15. The oligonucleotide of claim 5, wherein Q is a sequence selected from the group consisting of SEQ ID NOs: 1-70; a is 0; and b is 1.

16. A composition comprising:
(i) a safe and effective amount of an oligonucleotide according to the following Formula I:
5′-Xₐ-Q-Y_{b}-3′ Formula I
wherein:
X and Y are each independently selected from the group consisting of a lipid and a polyethylene glycol;
Q is a quadruplex-forming guanine-rich promoter gene oligonucleotide (GPGO);
a is 0 or 1; and
b is 0 or 1,
wherein the sum of a+b=1 or 2; and
(ii) a carrier,
wherein the oligonucleotide forms at least one quadruplex.

17. A method of inhibiting cell growth comprising contacting a cell with the composition according to claim 16.

18. A method of treating cancer comprising administering to a patient in need thereof the composition according to claim 16.

19. The method of claim 18, wherein the cancer is a cancer that expresses an oncogene selected from the group consisting of c-Myc, c-Myb, VEGF, Bcl-2, K-ras, HIF-1α, Rb, RET, c-Kit, and PDGF-A.

20. The method of claim 18, wherein the composition is administered orally, rectally, parenterally, intracistemally, intravaginally, intraperitoneally, intravesically, locally, or as a buccal or nasal spray.

21. A method of treating a patient having a tumor, the method comprising:
(a) performing a biopsy on the tumor;
(b) determining a gene expression profile of the tumor;
(c) identifying one or more oncogenes that are overexpressed in the tumor, based on the gene expression profile of step (b);
(d) administering to the patient a composition according to claim 16, wherein Q is a GPGO derived from the one or more overexpressed oncogenes identified in step (c).

22. The method of claim 21, wherein Q is a GPGO derived from an oncogene selected from the group consisting of c-Myc, c-Myb, VEGF, Bcl-2, K-ras, HIF-1α, Rb, RET, c-Kit, and PDGF-A.

23. The method of claim 22, wherein Q is a sequence selected from the group consisting of SEQ ID NOs: 1-70.


SEQUENCE LISTING (70 sequences)

SEQ ID NO: 1 - DNA, 27 bp, Homo sapiens
  misc_feature: c-myc
tggggagggtggggagggtggggaagg

SEQ ID NO: 2 - DNA, 23 bp, Homo sapiens
  misc_feature: c-myc
gggagggtggggagggtggggaa

SEQ ID NO: 3 - DNA, 20 bp, Homo sapiens
  misc_feature: c-myc
gagggtggggagggtgggga

SEQ ID NO: 4 - DNA, 19 bp, Homo sapiens
  misc_feature: c-myc
gagggtggggagggtgggg

SEQ ID NO: 5 - DNA, 20 bp, Homo sapiens
  misc_feature: c-myc
ggagggtggggagggtgggg

SEQ ID NO: 6 - DNA, 15 bp, Homo sapiens
  misc_feature: c-myc
gggtggggagggtgg

SEQ ID NO: 7 - DNA, 13 bp, Homo sapiens
  misc_feature: c-myc
gggtggggagggt

SEQ ID NO: 8 - DNA, 11 bp, Homo sapiens
  misc_feature: c-myc
gtggggagggt

SEQ ID NO: 9 - DNA, 7 bp, Homo sapiens
  misc_feature: c-myc
ggggagg

SEQ ID NO: 10 - DNA, 21 bp, Homo sapiens
  misc_feature: c-myc
gggtggggagggtggggaagg

SEQ ID NO: 11 - DNA, 21 bp, Homo sapiens
  misc_feature: c-myc
tggggagggtggggagggtgg

SEQ ID NO: 12 - DNA, 20 bp, Homo sapiens
  misc_feature: c-myc
tggggagggtggggagggtg

SEQ ID NO: 13 - DNA, 20 bp, Homo sapiens
  misc_feature: c-myc
gggtggggagggtggggaag

SEQ ID NO: 14 - DNA, 16 bp, Homo sapiens
  misc_feature: c-myc
tggggagggtggggag

SEQ ID NO: 15 - DNA, 42 bp, Homo sapiens
  misc_feature: c-myb
aggaggaggaggaagaggaggaggaggcactggaggaggagg

SEQ ID NO: 16 - DNA, 38 bp, Homo sapiens
  misc_feature: c-myb
gaggaggaggaagaggaggaggaggcactggaggagga

SEQ ID NO: 17 - DNA, 34 bp, Homo sapiens
  misc_feature: c-myb
ggaggaggaagaggaggaggaggcactggaggag

SEQ ID NO: 18 - DNA, 30 bp, Homo sapiens
  misc_feature: c-myb
aggaggaagaggaggaggaggcactggagg

SEQ ID NO: 19 - DNA, 24 bp, Homo sapiens
  misc_feature: c-myb
gaggaagaggaggaggaggcactg

SEQ ID NO: 20 - DNA, 20 bp, Homo sapiens
  misc_feature: c-myb
ggaagaggaggaggaggcac

SEQ ID NO: 21 - DNA, 37 bp, Homo sapiens
  misc_feature: VEGF
gggggcgggccgggggcggggtcccggcggggcggag

SEQ ID NO: 22 - DNA, 33 bp, Homo sapiens
  misc_feature: VEGF
gggcgggccgggggcggggtcccggcggggcgg

SEQ ID NO: 23 - DNA, 31 bp, Homo sapiens
  misc_feature: VEGF
gggcgggccgggggcggggtcccggcggggc

SEQ ID NO: 24 - DNA, 25 bp, Homo sapiens
  misc_feature: VEGF
gggccgggggcggggtcccggcggg

SEQ ID NO: 25 - DNA, 21 bp, Homo sapiens
  misc_feature: VEGF
gccgggggcggggtcccggcg

SEQ ID NO: 26 - DNA, 17 bp, Homo sapiens
  misc_feature: VEGF
cgggggcggggtcccgg

SEQ ID NO: 27 - DNA, 32 bp, Homo sapiens
  misc_feature: K-ras
agggcggtgtgggaagagggaagagggggagg

SEQ ID NO: 28 - DNA, 28 bp, Homo sapiens
  misc_feature: K-ras
ggcggtgtgggaagagggaagaggggga

SEQ ID NO: 29 - DNA, 26 bp, Homo sapiens
  misc_feature: K-ras
gcggtgtgggaagagggaagaggggg

SEQ ID NO: 30 - DNA, 25 bp, Homo sapiens
  misc_feature: K-ras
cggtgtgggaagagggaagaggggg

SEQ ID NO: 31 - DNA, 24 bp, Homo sapiens
  misc_feature: K-ras
cggtgtgggaagagggaagagggg

SEQ ID NO: 32 - DNA, 22 bp, Homo sapiens
  misc_feature: K-ras
ggtgtgggaagagggaagaggg

SEQ ID NO: 33 - DNA, 20 bp, Homo sapiens
  misc_feature: K-ras
gtgtgggaagagggaagagg

SEQ ID NO: 34 - DNA, 16 bp, Homo sapiens
  misc_feature: K-ras
gtgggaagagggaaga

SEQ ID NO: 35 - DNA, 12 bp, Homo sapiens
  misc_feature: K-ras
gggaagagggaa

SEQ ID NO: 36 - DNA, 27 bp, Homo sapiens
  misc_feature: RET
gggggcggggcggggcggggatgggcg

SEQ ID NO: 37 - DNA, 23 bp, Homo sapiens
  misc_feature: RET
gggcggggcggggcggggatggg

SEQ ID NO: 38 - DNA, 19 bp, Homo sapiens
  misc_feature: RET
gcggggcggggcggggatg

SEQ ID NO: 39 - DNA, 15 bp, Homo sapiens
  misc_feature: RET
ggggcggggcgggga

SEQ ID NO: 40 - DNA, 11 bp, Homo sapiens
  misc_feature: RET
ggcggggcggg

SEQ ID NO: 41 - DNA, 21 bp, Homo sapiens
  misc_feature: HIF-1a
gggcgggggagaggggagggg

SEQ ID NO: 42 - DNA, 17 bp, Homo sapiens
  misc_feature: HIF-1a
gcgggggagaggggagg

SEQ ID NO: 43 - DNA, 16 bp, Homo sapiens
  misc_feature: HIF-1a
gcgggggagaggggag

SEQ ID NO: 44 - DNA, 16 bp, Homo sapiens
  misc_feature: HIF-1a
cgggggagaggggagg

SEQ ID NO: 45 - DNA, 15 bp, Homo sapiens
  misc_feature: HIF-1a
gcgggggagagggga

SEQ ID NO: 46 - DNA, 13 bp, Homo sapiens
  misc_feature: HIF-1a
gggggagagggga

SEQ ID NO: 47 - DNA, 9 bp, Homo sapiens
  misc_feature: HIF-1a
gggagaggg

SEQ ID NO: 48 - DNA, 48 bp, Homo sapiens
  misc_feature: PDGF-A
ggaggcgggggggggggggcgggggcgggggcgggggaggggcgcggc

SEQ ID NO: 49 - DNA, 44 bp, Homo sapiens
  misc_feature: PDGF-A
aggcgggggggggggggcgggggcgggggcgggggaggggcgcg

SEQ ID NO: 50 - DNA, 40 bp, Homo sapiens
  misc_feature: PDGF-A
gcgggggggggggggcgggggcgggggcgggggaggggcg

SEQ ID NO: 51 - DNA, 36 bp, Homo sapiens
  misc_feature: PDGF-A
gggggggggggggcgggggcgggggcgggggagggg

SEQ ID NO: 52 - DNA, 32 bp, Homo sapiens
  misc_feature: PDGF-A
gggggggggggcgggggcgggggcgggggagg

SEQ ID NO: 53 - DNA, 28 bp, Homo sapiens
  misc_feature: PDGF-A
gggggggggcgggggcgggggcggggga

SEQ ID NO: 54 - DNA, 24 bp, Homo sapiens
  misc_feature: PDGF-A
gggggggcgggggcgggggcgggg

SEQ ID NO: 55 - DNA, 20 bp, Homo sapiens
  misc_feature: PDGF-A
gggggcgggggcgggggcgg

SEQ ID NO: 56 - DNA, 41 bp, Homo sapiens
  misc_feature: Bcl-2
aggggcgggcgcgggaggaagggggcgggagcgggggctgg

SEQ ID NO: 57 - DNA, 37 bp, Homo sapiens
  misc_feature: Bcl-2
gggcgggcgcgggaggaagggggcgggagcgggggct

SEQ ID NO: 58 - DNA, 33 bp, Homo sapiens
  misc_feature: Bcl-2
gcgggcgcgggaggaagggggcgggagcggggg

SEQ ID NO: 59 - DNA, 29 bp, Homo sapiens
  misc_feature: Bcl-2
gggcgcgggaggaagggggcgggagcggg

SEQ ID NO: 60 - DNA, 25 bp, Homo sapiens
  misc_feature: Bcl-2
gcgcgggaggaagggggcgggagcg

SEQ ID NO: 61 - DNA, 23 bp, Homo sapiens
  misc_feature: Bcl-2
gggcgcgggaggaagggggcggg

SEQ ID NO: 62 - DNA, 21 bp, Homo sapiens
  misc_feature: Bcl-2
gcgggaggaagggggcgggag

SEQ ID NO: 63 - DNA, 19 bp, Homo sapiens
  misc_feature: Bcl-2
cgggaggaagggggcggga

SEQ ID NO: 64 - DNA, 18 bp, Homo sapiens
  misc_feature: Bcl-2
gggaggaagggggcggga

SEQ ID NO: 65 - DNA, 17 bp, Homo sapiens
  misc_feature: Bcl-2
gggaggaagggggcggg

SEQ ID NO: 66 - DNA, 31 bp, Homo sapiens
  misc_feature: c-Kit
cagagggagggcgctgggaggaggggctgct

SEQ ID NO: 67 - DNA, 27 bp, Homo sapiens
  misc_feature: c-Kit
gagggagggcgctgggaggaggggctg

SEQ ID NO: 68 - DNA, 23 bp, Homo sapiens
  misc_feature: c-Kit
gggagggcgctgggaggaggggc

SEQ ID NO: 69 - DNA, 19 bp, Homo sapiens
  misc_feature: c-Kit
gagggcgctgggaggaggg

SEQ ID NO: 70 - DNA, 15 bp, Homo sapiens
  misc_feature: c-Kit
gggcgctgggaggag